(19) United States
(12) Patent Application Publication
(10) Pub. No.: US 2008/0140164 A1
(43) Pub. Date: Jun. 12, 2008

(54) LIGHT EMITTING THERAPEUTIC DEVICES AND METHODS

(75) Inventors: Richard Oberreiter, Newport Beach, CA (US); James Harry Kraushaar, Newport Beach, CA (US)

Correspondence Address: KNOBBE MARTENS OLSON & BEAR LLP, 2040 MAIN STREET, FOURTEENTH FLOOR, IRVINE, CA, 92614, US

(73) Assignee: CLRS Technology Corporation, Newport Beach, CA (US)

(21) Appl. No.: 11/951,240

(22) Filed: Dec. 5, 2007

Related U.S. Application Data
(60) Provisional application No. 60/873,559, filed on Dec. 6, 2006.

(51) Int. Cl. A61N 5/06; A61B 18/18

(52) U.S. Cl. 607/88; 606/3; 606/9

(57) ABSTRACT

A light emitting device for providing therapy to a user includes a light source configured to generate optical energy having a wavelength in a range of from about 400 nm to about 1100 nm. The device further includes a user interface configured to be placed into contact with a treatment area on a user's body and configured to transmit the optical energy from the light source to the treatment area generally along a beam propagation axis. The user interface includes an electrical impedance sensor configured to determine when the user interface is contacting the treatment area. The device also includes a controller, configured to receive at least one sensor signal from the electrical impedance sensor, wherein the controller is configured to prevent activation of the light source based upon the at least one sensor signal.

### CROSS-REFERENCE TO RELATED APPLICATIONS

The present application claims priority from U.S. Provisional No. 60/873,559, filed Dec. 6, 2006, which is incorporated by reference herein.

### BACKGROUND

1. Field

The disclosure generally relates to devices for treating skin conditions. For example, with respect to some embodiments, the disclosure relates to light-emitting devices and methods for the treatment of skin conditions including acne.

2. Description of the Related Art

Skin conditions can cause serious health risk including scarring and psychological damage. One of the most common skin conditions is acne, the most common form being acne vulgaris. Acne affects millions of people in the United States and is an inflammatory disease caused generally as a result of blockages in hair follicles. Acne affects the face and upper neck most commonly, but other areas of the body may also develop acne blemishes. While acne most commonly affects people during adolescence, it can affect people of all ages.

There is significant demand for skin treatment devices, particularly for those that treat acne. Several acne treatment methods are known including topical bactericidal products, topical antibiotics, oral antibiotics, hormonal treatments, topical retinoids and oral retinoids. Less common treatment methods include the use azelaic acid, zinc, tea tree oil, nicotinamide, and other agents. However, these products often have undesirable side effects, or have limited results.

Devices have also been used to treat acne, but the equipment is often large, expensive and difficult to use. There is therefore a need for a safe, user-friendly, hand-held, light emitting therapeutic device to treat skin conditions including acne.

### SUMMARY

In one embodiment, a light emitting device for providing therapy to a user includes a light source configured to generate optical energy having a wavelength in a range of from about 400 nm to about 1100 nm. The device also includes a user interface configured to be placed into contact with a treatment area on a user's body and configured to transmit said optical energy from said light source to said treatment area generally along a beam propagation axis. The user interface includes an electrical impedance sensor configured to determine when said user interface is contacting said treatment area. The device also includes a controller, configured to receive at least one sensor signal from said electrical impedance sensor, wherein said controller is configured to prevent activation of said light source based upon said at least one sensor signal.

In various embodiments, the light source of may be a flashlamp or an LED. Moreover, the wavelength may be in a range of from about 400 nm to about 700 nm. The light emitting device may also include a transmission window configured to filter said optical energy prior to transmission to said treatment area.

The light emitting device may also include a second light source configured to generate second optical energy having a second wavelength. The second wavelength may be an infrared wavelength or a blue wavelength. The second light source may be an LED.

An aiming beam may also be included with the light emitting device, the aiming beam configured to illuminate the treatment area prior to activation of said light source. The light emitting device may further include a user input configured to activate said light source. The user input may include a button having a button depression axis, wherein said button depression axis is substantially aligned with said beam propagation axis. Said user interface may define a transmission pathway through an opening in said user interface, said user interface further including a locating ridge positioned at least partially around said opening and configured to provide tactile feedback to a user regarding the position of the opening. The locating ridge may extend around the entire opening.

In another embodiment, a light emitting device for providing therapy to a user includes a light source configured to generate optical energy having a wavelength in a range of from about 400 nm to about 1100 nm. The device further includes a user interface configured to be placed into contact with a treatment area on a user's body and configured to transmit said optical energy from said light source to said treatment area. The user interface defines a transmission pathway of said optical energy from said light source to said treatment area. The user interface includes a first contact sensor and a second contact sensor spaced apart from said first contact sensor. Moreover, a linear path from said first contact sensor to said second contact sensor at least partially traverses said transmission pathway.

The light emitting device may further include a controller configured to activate said light source only when both said first and second contact sensors are in contact with said treatment area. The first and second contact sensors may include first and second impedance sensors. Moreover, said light source may include a flashlamp. The device may further include a filter configured to filter said optical energy prior to delivery to said treatment area. Further, the device may include second light source configured to generate second optical energy having a second wavelength. The second light source may be an LED.

In another embodiment, a device light emitting device for providing therapy to a user includes a light source configured to generate optical energy having a wavelength in a range of from about 400 nm to about 1100 nm. The device further includes a user interface configured to be placed into contact with a treatment area on a user's body and configured to transmit said optical energy from said light source to said treatment area. The user interface may include an output window and at least two contact sensors. The device further includes a controller configured to determine an angular alignment between said output window and said treatment area prior to delivering optical energy to said treatment area.

The controller permits activation of said light source when said output window is determined to be substantially parallel to said treatment area. In one embodiment, said controller permits activation of said light source when said output window is determined to be inclined with respect to said treatment area no more than about 22 degrees.

The at least two contact sensors may include at least two impedance sensors. Moreover, the light emitting device may further include a second light source configured to generate blue light. The second light source may be an LED.

In another embodiment, a method of treating a physiological condition with optical energy includes providing a light emitting device, said light emitting device including a light source configured to generate optical energy having a wavelength in a range of from about 400 nm to about 1100 nm. The device further includes a user interface configured to provide a transmission pathway of said optical energy from said light source to a treatment area generally along a beam propagation axis. The user interface includes an electrical impedance sensor. The device also includes a controller which is in electrical communication with said light source and electrical impedance sensor. The method further includes generating an impedance signal with said electrical impedance sensor and preventing generation of said optical energy when said impedance signal indicates that said user interface is not in contact with said treatment site.

Moreover, the method may further include generating said optical energy and directing said optical energy to said treatment area. The method may also further include filtering said optical energy prior to delivery to said treatment area. The filtering may remove energy having a wavelength outside of a range of from about 400 nm to about 700 nm.

The method may further include generating second optical energy with a second light source. The said second optical energy may include mostly infrared energy. The method may also include illuminating said treatment area with an illumination light source.

In another embodiment, a method of treating a physiological condition with optical energy includes providing a light emitting device. The light emitting device includes a light source configured to generate optical energy having a wavelength in a range of from about 400 nm to about 1100 nm. The device further includes a user interface configured to provide a transmission pathway of said optical energy from said light source to a treatment area. The user interface includes a first contact sensor and a second contact sensor spaced apart from said first contact sensor. A linear path from said first contact sensor to said second contact sensor at least partially traverses said transmission pathway. The method further includes determining an contact signal with said contact sensors, receiving a user input to activate said light source, and activating said light source in response to said contact signal and user input.

The activating step may include activating said light source when said contact signal indicates that said user interface is in contact with said treatment area and when said user input is activated. Receiving a user input may include determining if a button has been pressed or released.

The method may further include filtering said optical energy after activating said light source. The filtering step may remove energy having a wavelength outside of a range of from about 400 nm to about 700 nm. The method may further include generating second optical energy with a second light source. Moreover, said second optical energy may include mostly infrared energy. Finally, the method may further include illuminating said treatment area with an illumination light source prior to said activating.

In another embodiment, a method of treating a physiological condition with optical energy includes providing a light emitting device. The device includes a light source configured to generate optical energy having a wavelength in a range of from about 400 nm to about 1100 nm. The device further includes a user interface, configured to transmit said optical energy from said light source to said treatment area. The user interface includes an output window, and at least two sensors. The method further includes receiving at least two sensor signals from said at least two sensors and determining an angular alignment between said output window and said treatment area based upon said at least two sensor signals.

The method may further include enabling activation of said light source in response to said angular alignment. The enabling step may occur only when said angular alignment indicates that said output window and said treatment area are substantially parallel. Moreover, said enabling may occur only when said angular alignment indicates that said output window and said treatment area are substantially in contact.

The method may further include illuminating said treatment area with an illumination light source where said light emitting device further include said illumination light source.

For purposes of summarizing the invention, certain aspects, advantages and novel features have been described herein. Of course, not necessarily all such aspects, advantages or features will be embodied in any particular embodiment.

### BRIEF DESCRIPTION OF THE DRAWINGS

These and other features will now be described with reference to the drawings summarized below. These drawings and the associated description are provided to illustrate certain embodiments, and not to limit the scope of the invention.

FIG. 1 is a block diagram of an acne treatment device according to an embodiment of the disclosure;

FIGS. 2A-C are front perspective, rear perspective and right side views, respectively, of an acne treatment device according to an embodiment of the disclosure;

FIGS. 3A-B are right side and top views, respectively, of an acne treatment device according to an embodiment of the disclosure;

FIGS. 4A-E are front perspective, right side, and three front perspective views, respectively, of several acne treatment devices according to various embodiments of the disclosure;

FIGS. 5A-B are cross-sectional views of output interfaces of an acne treatment device according to embodiments of the disclosure;

FIGS. 6A-E are front views of the head portion of an acne treatment device including safety mechanisms including sensor arrays according to various embodiments of the disclosure; and

FIGS. 7-10 show various embodiments of methods of treating acne with an acne treatment device according to various embodiments of the disclosure.

### DETAILED DESCRIPTION

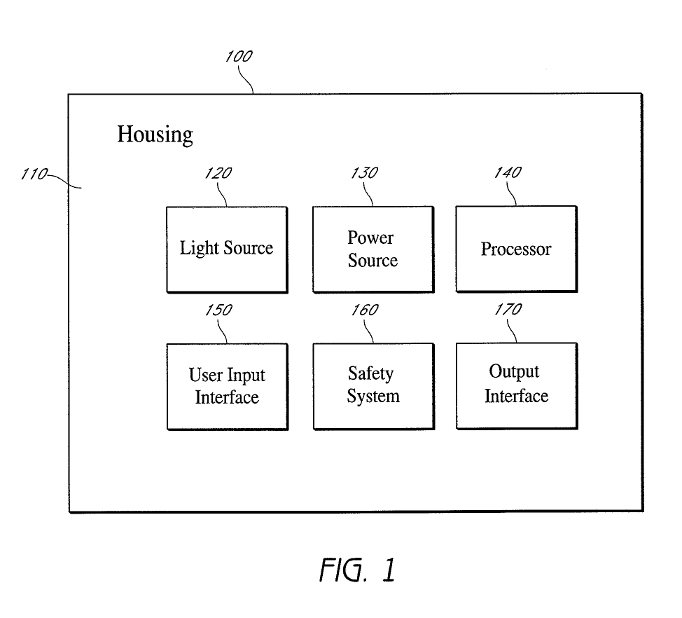

A light emitting therapeutic device 100 in accordance with one embodiment of the present disclosure is illustrated in FIG. 1. In one embodiment, the device 100 is a hand-held, ergonomically designed unit that allows a user to treat him or herself. The device 100 may also be described as a self-contained, hand-held, portable unit that is configured to be carried by a user. For example, in various embodiments, the device 100 is configured to be carried in the user's pant, shirt, or jacket pocket, or within a purse, handbag, or backpack.

In the illustrated embodiment, the device 100 includes a housing 110 that contains the device's internal components. The mechanical and electronic parts used to operate the device 100 are contained within the housing 110. In the illustrated embodiment, a light source 120, power source 130, processor 140 (sometimes referred to as a controller 140), user input interface 150, safety system 160, and output interface 170 are carried by or contained within the housing 110 of device the 100.

In some embodiments, light generated by the light source 120 and emitted from the device 100 has a wavelength configured or selected to penetrate the outer layers of skin sufficiently to cause a photo-dynamic effect that kills the P. Acnes bacteria and thereby treats acne. P. Acnes bacteria is one cause of acne. By destroying the bacteria, the device 100 removes acne blemishes from a user's skin. In some embodiments, light emitted from the device 100 penetrates the outer layers of the skin causing a thermal effect that kills the P. Acnes bacteria. In some embodiments, the photo-dynamic effect is the primary effect that leads to killing the P. Acnes bacteria. In other embodiments, the thermal effect is the primary effect. In yet other embodiments, the photo-dynamic and thermal effects are relatively equal.

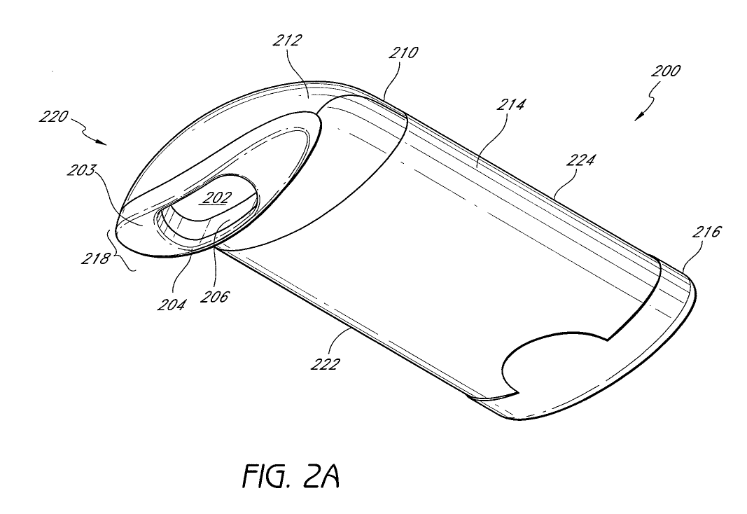

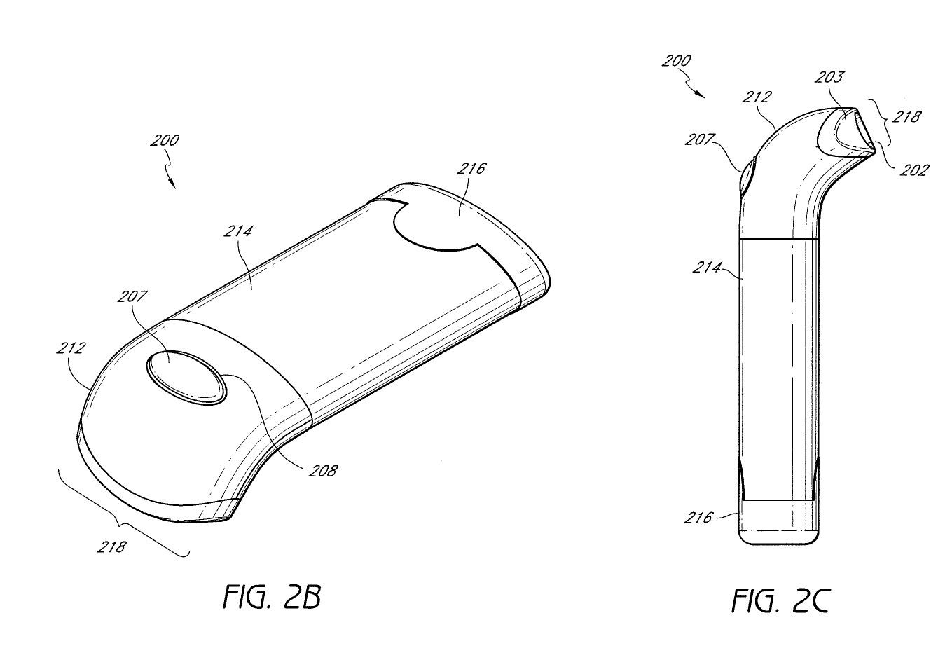

FIGS. 2A-C illustrate another embodiment of the device 100. In some embodiments, the device 200 is the same as, or includes one or more of the same components of the device 100 described above with respect to FIG. 1. The device 200 includes a housing 210, which is configured to hold the device components. For example, the housing 210 can hold a light source, power source, controller, user interface, safety system, and/or output interface.

The housing 210 includes a head portion 212, a body portion 214, and a base portion 216. An output interface 218 (sometimes referred to as a user interface 218), is coupled to the hosing 210 at the device's light-emitting end 220. In one embodiment, the portions 212, 214, 216 are removably attachable to one another. In other embodiments, the housing 210 is a single, molded, contiguous piece.

In other embodiments, the output interface 218, or portions thereof, are removably attachable to the housing 210. The removably attachable portions may be removed by using any of a variety of mechanisms, including friction mechanisms, such as friction locks, snaps, sliders, ridges, threads, etc., as well as other mechanical devices, including screws, locks, rings, etc.

The various segments or portions thereof that are removably attachable may be disposable. For example, the entire head portion 212, output interface 218, or portions thereof are disposable in some embodiments.

The housing 210 is ergonomically shaped and helps avoid fatigue during use. In some embodiments, for example the body portion 214 forms a handle region large enough accommodate a user's hand comfortably while allowing a firm grip. Moreover, in the illustrated embodiment, the sides 222, 224 of the device 200 are rounded to comfortably accommodate the users thumb and fingers when gripping the device 200.

The housing 210 may be made of, for example, various types of metal, plastic, rubber, or a combination thereof. In some embodiments, the segments 212, 214, 216 of the housing 210 are made from different materials. For example, in some embodiments, the head portion 212 is made from plastic and the body portion 214 is made of metal, or vice versa.

In one embodiment, the device 200 includes a controller (not shown), such as the processor 140 described above with respect to FIG. 1. In one embodiment, the controller (or processor 140) is made from discrete logic only, and does not include a microprocessor or microcontroller. In such embodiments, the device 200 does not include any software or firmware. This advantageously helps simplify the electronics, reduces costs, and can greatly simply design validation as well as regulatory review by agencies such as the Food & Drug Administration (the FDA).

In other embodiments, the controller (or processor 140) includes a controller, microcontroller, or memory, including a PIC microcontroller, embedded logic, a ROM, an EPROM, an EEPROM, a field-programmable gate array (FPGA), firmware or other programmable logic device (PLD). In other embodiments the controller (or processor 140) includes and ASIC, a soft microprocessor, or a complex programmable logic device (CPLD).

The controller controls operation of the device 200, as discussed in greater detail below. In general, wherever operation of the device 200 is discussed below, the controller may be the component which implements the operation even if not specifically mentioned with respect to the described operation.

In various embodiments, the controller includes a general purpose, single-chip or multi-chip microprocessor (such as a Pentium® processor, a Pentium® II processor, a Pentium® Pro processor, an xx86 processor, an 8051 processor, a MIPS® processor, a Power PC® processor, or an ALPHA® processor). In addition, the controller may include a special purpose microprocessor, such as a digital signal processor.

The device 200 also includes a power source (not shown), which in some embodiments is the same as the power source 150 described above with respect to FIG. 1. The power source provides the power to operate the device 200. In some embodiments, the power source includes a battery (such as a disposable or a rechargeable battery), a power cell, a fuel cell, and/or a capacitor. In some embodiments, the power source includes a single capacitor capable of holding the entire charge needed to power the device 200. In other embodiments, multiple, smaller capacitors are used. The power source generally includes capacitor charging and light source triggering circuitry, as well.

The power source may be physically located in any portion of the device 200. For example, in one embodiment, the power source is located in the base portion 216, which advantageously provides a counterbalance to the weight of the output interface 218 and provides easy access to a user. In one embodiment, the power source is disposable, such as a disposable battery. In other embodiments, the power source 130 is rechargeable, such as a lead and sulfuric acid, nickel cadmium (NiCd), nickel metal hydride (NiMH), lithium ion (Li-ion), or a lithium ion polymer (Li-ion polymer) battery).

The device 200 also includes a light source (not shown), which in some embodiments is the same as the light source 120 described above with respect to FIG. 1. In some embodiments, the light source is configured to emit a broad spectrum light. For example, the light source can include a flashlamp, such as a xenon or krypton gas filled flashlamp, or other broadband light source. Broadband light sources can be configured to emit light having wavelengths in the range of from about 400 nm to about 1100 nm.

In other embodiments, the light source is configured to emit monochromatic or substantially monochromatic light. For example, the light source can include an LED, diode, laser, or other narrow-band light source. In yet other embodiments, broad spectrum and monochromatic light is combined from one or more light sources, or alternated in their application from the device 200. In other embodiments, light of multiple wavelengths is emitted simultaneously or sequentially.

The output from the light source can be controlled or modulated prior to delivery to the user. For example, in some embodiments, the light emitted from the light source is passed through a filter. In one embodiment, the filtered light has a wavelength greater than 400 nm. The filtered light can have wavelengths in a range from about 400 nm to about 1100 nm, or from about 400 nm to about 700 nm. In other embodiments, the filtered light has a wavelength mostly at around 400 nm. The filter can be provided as an optical coating to the light source (e.g., flashlamp), or as a window positioned between the light source and the user's treatment site.

In addition, the optical characteristics of the light generated by the light source can be controlled by the controller, or other electronic circuitry included with the housing 210. For example, by pulsing the light source, the emitted light's pulse shape can be modulated and controlled. In addition, by varying the drive current and/or voltage to the light source, the output power can be modulated or controlled.

In one embodiment, light emitted from the light source has a wavelength of about 400 nm. In other embodiments, the wavelength is greater than 400 nm. For example, in some embodiments, the wavelength is between about 400 and 700 nm. In other embodiments, the wavelength may be between 400 and 1100 nm. In some embodiments, the wavelength may be greater than 1100 nm. In one embodiment, the wavelength is in the blue spectrum, and the light emitted from the light source 120 is blue light.

As discussed above, the particular wavelength or range of wavelengths directed to a treatment site on a user can be controlled by using a narrow band light source configured to emit light at a desired wavelength (or range of wavelengths), or by using a broad band light source with filters to filter out or remove undesired light wavelengths.

In some embodiments, the light source includes both a broadband light source and a narrowband light source. For example, in one embodiment, the light source includes a flashlamps and one or more light emitting diodes (LEDs).

In another embodiment, the light source includes two flash lamps, each having a different optical coating configured to filter out different wavelengths. For example, in one embodiment, one coating is designed to transmit light in the infrared spectrum (or a portion thereof), and the other coating is designed to transmit light corresponding to the visible blue spectrum (or a portion thereof). In another embodiment, the two flashlamps are each housed in a different chamber within the housing 210, each chamber having a different filter window at its chamber output. In other embodiments, only one primary wavelength of light is transmitted through the optical coating, while in other embodiments, light having multiple primary wavelengths is transmitted therethrough.

In some embodiments, the wavelength or wavelengths transmitted to a treatment site are selectable; either automatically by the device 200 itself, or by the user via a user interface (not shown). For example, in some embodiments, a user can select a desired treatment wavelength from a range using, for example, a series of buttons or a dial corresponding to a variety of wavelength ranges. Alternatively, the device 200 can include a digital user interface which allows a user to select a wavelength range or ranges from a menu displayed on a display. In some embodiments, the user selection will cause different types of optical filters to be placed in the path of the light source's output. In some embodiments, for example, a dial is mechanically coupled to an optical filter having different filtering materials on different sections, and turning the dial causes the filter material placed in the light path to change. In other embodiments, user selection causes the controller to actuate a motor or other device to move the desired filter into place.

The optical characteristic of the light emitted by light source may be varied as a function of skin pigmentation. For example, the variation may be based on the Fitzpatrick Classification Scale of skin pigmentation types. For example, when the user indicates that the device 200 is going to be used to treat a darker skin types, the controller will control the light source to generate pulsed optical energy having a longer pulse duration than when lighter skin type treatment is selected.

The optical characteristic may be automatically selected by the controller based on a user selection of skin pigmentation type, or by sensing the treatment site skin pigmentation. For example, the device 200 may include a dial or other form of user input interface to allow a user to set their skin pigmentation type. The controller could then, based on the user's skin pigmentation type, select the appropriate treatment pulse duration, peak power, average power, pulse interval, duty cycle, etc., and would actuate the flash lamp accordingly.

In other embodiments, the user selects the optical characteristic directly instead of selecting their pigmentation type and using the controller to determine optical characteristic. In yet other embodiments, the device 200 includes a colorimeter or other device to automatically determine the pigmentation of a user's treatment site. Furthermore, light emitted from the light source is sometimes characterized as an intense pulse of light. In some embodiments, the light source is removable from the device 200 by the user, either by hand, or with a tool.

In some embodiments, the light source includes a reflector to reflect, direct, and/or focus energy emitted from the light source towards the patient's skin. The reflector increases the amount of light received by the tissue and thereby increases the photo-therapeutic effect. In some embodiments the reflector has a parabolic cross sectional shape and extends along substantially the entire length of the light source. In other embodiments the reflector has a concave cross section.

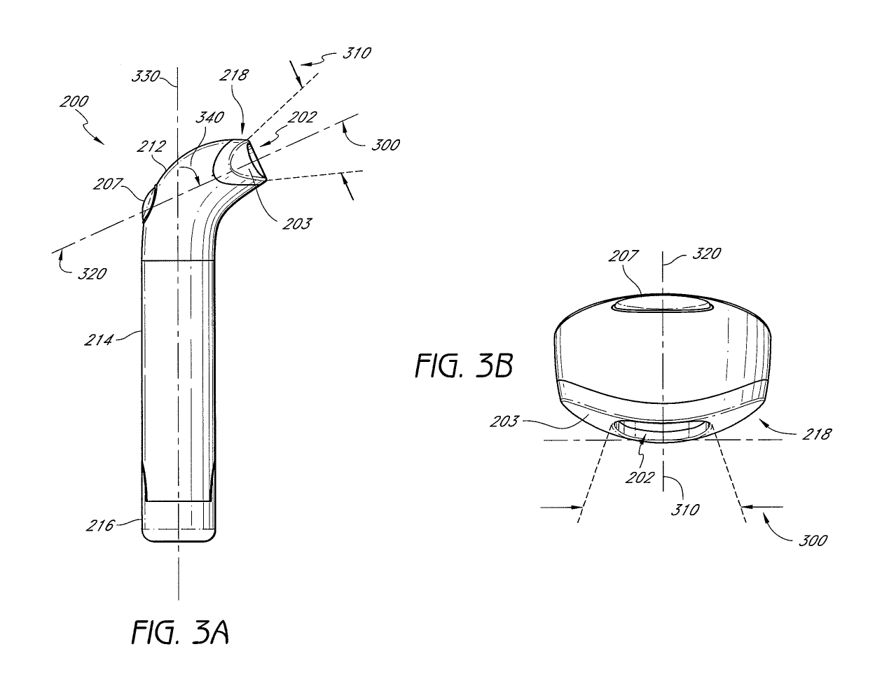

Referring now to FIG. 3, in one embodiment, light generated by the light source is configured to travel along a beam propagation axis 300 as it travels through the transmission path 202 (sometimes referred to as a transmission channel 202, emission path 202, or emission channel 202) defined by the user interface 218. The light diverges as it travels along the propagation axis 300, thereby defining a beam propagation envelope 310.

Because the light diverges as it travels along the propagation axis 300, the light's energy density decreases as the distance from the light source to the treatment site increases. Therefore, to maximize energy density, the user interface 218 of the device 200 is brought into contact with the user's treatment site prior to activating the light source. When properly oriented, light generated by the light source will have an energy density in the range of from about 1 J/cm² to about 3 J/cm² (or from 1 J/cm² to 3 J/cm²) at the treatment site. In other embodiments, the energy density is in the range of about 1 J/cm² to about 10 J/cm² (or from 1 J/cm² to 10 J/cm²) In one embodiment, the energy density is about 6 J/cm².

In addition, in some embodiments, the light source delivers peak optical pulse power in the range of from about 5 kW to about 20 kW (or from 5 kW to 20 kW). In some embodiments, the light spot at the treatment site has an area of about 1 cm². In general, the light generated by the light source is safe to use near human eyes and will not cause serious or irreparable harm to the structures of the eye if it is accidentally discharged near or into the eye.

Referring to FIGS. 2A-3B, in one embodiment, the acne treatment device 200 also includes a user input 207 (which in some embodiments is the same as the user input interface 150 described above with respect to FIG. 1). The user input 207 for user control of various operational features. For example, in various embodiments, the user input 207 includes a switch, button (as illustrated), contact, and/or sensor. In some embodiments, the user input 207 causes the device 200 to turn on and/or off, to charge a power supply, to begin or end light flashing, to program the exposure duration, and/or to enter a code to re-activate the device 200.

In the illustrated embodiment, button 207 causes the light source to first charge and then flash. In some embodiments there is a separate power mechanism such as a button or switch to turn on the device 200, while in other embodiments, a single button turns and activates the device 200.

For example, in some embodiments, the user presses the button 207 once in order to power on the device 200, which causes the flashing circuitry to charge. While the circuitry is charging, a status indicator 208 indicates that the device 200 is not ready to be activated. For example, during charging, the status indicator can illuminate to a red color. Once the flashing circuitry is charged, the status indicator 208 changes color to indicate that the device 200 is ready for use. For example, the status indicator changes to a green color.

Once charged and ready, pressing and releasing the button 207 cause the light source to flash and emit light. When the light source flashes, the flashing circuitry discharges through the light source, and the status indicator 208 again indicates that the device is not ready to be activated. The process can be repeated to deliver additional optical energy to a treatment site, or the device 200 may then be turned off. For example, the device 200 may be turned off by pressing and holding the button 207 for a specified duration.

In other embodiments, the user holds the button 207 to fully charge the flashing circuitry, e.g., until the status indicator 208 changes color from red to green. If the user releases the button prior to fully charging the flashing circuitry, the flashing circuitry will discharge, and light will not be generated from the light source. But if the flashing circuitry becomes fully charged, pressing (or pressing and releasing) the button 207 again causes the light source to activate.

In some embodiments, the device 200 includes an illumination light source (not shown) in addition to the therapeutic light source discussed above. The illumination light source may be located in or around the output interface 218 or on another portion of device 200. The illumination light source illuminates portions of the patient's skin in order to identify blemishes and problem areas for therapeutic treatment. The illumination source, for example, may include one or more LEDs, such as white light emitting LEDs. In some embodiments, the device 200 also includes an aiming or pointing mechanism (not shown), such as an aiming beam, or a laser pointer. The aiming mechanism allows the user to more accurately identify problem areas and position and orient the device 200 prior to treatment.

In some embodiments the device 200 includes an aiming beam, an illumination source, or both. The aiming beam or illumination source may be activated by a separate button. For example, in some embodiments a button may be located on the side 222, 224 of the body portion 214 or head portion 212, such that it is accessible by the user's thumb. The user may activate the aiming beam or illumination source by depressing the button on the side with the thumb. Then the user may activate flash the device 200 using the button 207. In other embodiments, a partial depression of button 207 activates the aiming beam or illumination source and a full depression causes the device 200 to charge the flashing circuitry and activate the therapeutic light source.

Various aspects of the device 200 design provide intuitive use, and help the user orient the output interface 218 prior to activating the light source. This can be important for users that do not have access to a mirror during device 200 usage. For example, the arrangement of the button 207 with respect to the output interface 218 allows for beam propagation axis alignment with respect to a treatment area.

Referring again to FIGS. 3A and 3B, the button 207 may be pressed such that it moves along a button depression axis 320. The button depression axis 320 is substantially aligned with the beam propagation axis 310. This configuration advantageously allows a user to align the beam propagation axis with a treatment site by simply pointing at a desired treatment site or area with the finger used to press the button 207. Once aligned, the user may press the button 207 and to activate the light source, and to cause light from the light source to be directed to the treatment area.

Moreover, the general shape of device 200 allows for intuitive and ergonomic application of treatment. For example, the head portion's angulation 340, defined by the device's longitudinal axis 330 and beam propagation axis 300, allows for natural alignment of the users wrist and fingers when applying treatment to most areas of the body.

As discussed above, the device 200 includes an output interface 218 that serves as the interface between the device 200 and the treatment site, e.g., the user's skin. The output interface 218 can include a transmission surface, mirror, and/or window. In one embodiment, the output interface 218 is disposable. In other embodiments, portions of the output interface 218 are disposable.

In the illustrated embodiment of FIGS. 2A, 2C, 3A, and 3B, the output interface 218 includes a surface 203 which is substantially orthogonal to the beam propagation axis 310. The surface 203 may contact the patient's skin and may be made of metal or plastic material. In some embodiments, the surface 203 is made of a soft rubber. In some embodiments, the surface 203 may be smooth so as to glide across the patient's skin.

The output interface 218 also includes an emission channel 202 through which the therapeutic light is emitted. As shown the emission channel 202 may have an oval shape cross-sectional shape. In other embodiments, the emission channel 202 may have a circular or rectangular cross-sectional shape.

In the illustrated embodiment, the therapeutic light generated by the light source travels through a transmission window 206 prior to exiting from the device 100. In some embodiments, the transmission window 206 is recessed from an output rim 204 as defined by emission channel 202 and surface 203. The transmission window 206 may be made of various optically-transparent materials including glass, quartz, fluorite or plastic, such as acrylic. The transmission window 206 may include an optical lens which refracts the emitted light to focus it onto a treatment area. Alternatively, the transmission window 206 may have a planar surface. In other embodiments, the transmission window 206 includes an optical coating to filter out undesirable wavelengths from broadband light generated by the light source.

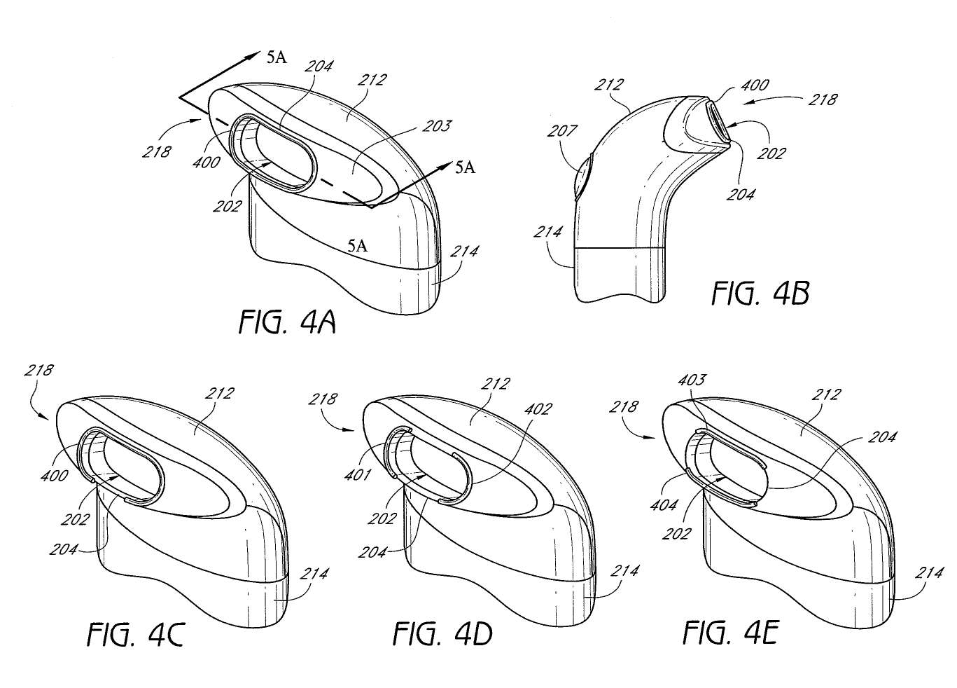

Referring now to FIGS. 4A-E, in some embodiments, the output interface 218 includes a locating ridge 400 that extends from the surface 203 in the general direction of the beam propagation. As illustrated by FIG. 4A, the locating ridge 400 may be shaped to conform to the shape of the rim 204 defined by the emission channel 202. In one embodiment, the locating ridge 400 is made of a soft material, such as rubber, nylon, polyethylene, and/or expanded polytetrafluoroethylene (ePTFE). Certain materials, such as ePTFE, have low friction, lubricious qualities, that provide enhanced comfort to a user when placing the output interface 218 in contact with, and when moved against, the user's skin. The locating ridge 400 can be made from a soft material that conforms to the patient's features and/or blemishes as the device 200 moves across the skin. In other embodiments, the locating ridge 400 is made of a plastic or metal. In one embodiment, the locating ridge 400 is made of an opaque material. The locating ridge 400 may therefore serve to increase the level of comfort associated with using the device 200, and also to act as a shield to limit, reduce, or prevent light from reaching the eyes of the user or others.

As shown in FIGS. 4A-C, the locating ridge 400 may be one contiguous member. In other embodiments, as illustrated by FIGS. 4D and 4E, the locating ridge 400 may include multiple segments 401, 402, 403, 404. The embodiments illustrated in FIGS. 4D-E show configurations having two segments 401, 402 disposed vertically on the sides of the emission channel 202, and two segments 403, 404 disposed horizontally on the top and bottom of the emission channel 202, respectively. However, in other embodiments, there may be more than two segments arranged in different configurations. In one embodiment, locating ridge segments are provided on opposite sides of the emission channel 202.

As illustrated by FIG. 4A, the locating ridge 400 may extend along the entire rim 204 of the emission channel 202. Alternatively, in other embodiments, such as illustrated in FIG. 4C-E, the segment or segments may only cover or extend along a portion of the rim 204, leaving an opening. The opening or openings may serve to increase the level of comfort associated with using the device 200 by limiting the contact of the locating ridge 400 with sensitive blemishes when the device 200 is moved across the user's skin.

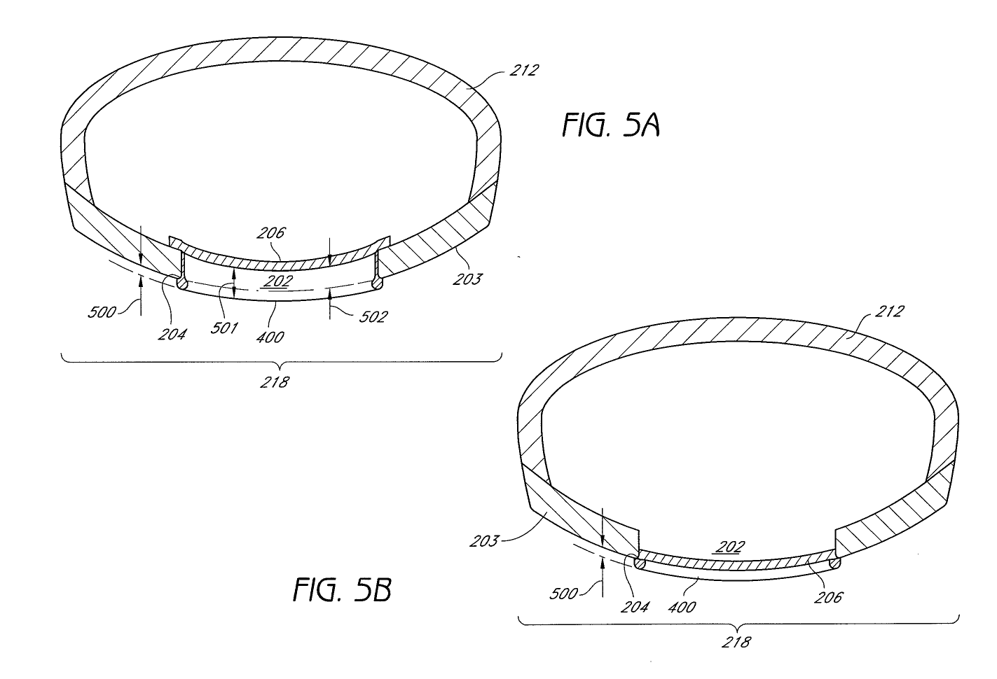

As illustrated by FIG. 5A, the transmission window 206 may be recessed or set back from surface 203 of the user interface 218 by a predetermined distance 502. In addition, the locating ridge 400 protrudes from output surface 203 a predetermined protrusion distance 500. When configured in this manner, the transmission window 206 is set back from the user's skin a total distance 501, which equals the sum of the predetermined distance 502 and the predetermined protrusion distance 500, when the device 200 is used.

In another embodiment, the locating ridge 400 is not provided, and the total distance 501 from the transmission window 206 to the user's skin (when the device 200 is used) is simply the predetermined distance 502. In yet other embodiments, as illustrated in FIG. 5B, the transmission window may be aligned flush with surface 203. As such, the total distance 501 from the transmission window 206 to the user's skin (when the device 200 is used) is simply the predetermined protrusion distance 500. In the configuration illustrated by FIG. 5B, if no locating ridge 400 is present, transmission window is not set back from the user's skin at all, and makes direct contact to the user's skin (when the device 200 is used).

The arrangement of the output surface 203, the transmission window 206, the output rim 204 and the locating ridge 400 alone or together provide tactile information to the user when the device 200 is used. Tactile information advantageously helps the user determine the position and orientation of the device 200 prior to and during use.

In one embodiment, the emission channel 202 defines a large enough application area to completely surround the acne. In one embodiment, for example, the application area is 1 cm². Moreover, the user interface 218 may define one or more distances 500, 501, 502 between the surface 203 and transmission window 206 deep enough to envelope most blemishes. As the device 200 is moved across the user's skin the user will feel when blemishes are surrounded by the rim 204 and/or contained in the space defined by locating ridge 400. As such, the user will know when the device 200 is properly positioned with respect to a particular blemish to deliver a therapeutic treatment.

In some embodiments, the output interface 218 and/or various portions thereof (including the locating ridge 400) are removably attachable and or disposable. In some embodiments, for example, friction mechanisms may be used to allow for removable attachment. In other embodiments, latching mechanisms such as a latch and pocket type of mechanism may be employed. In other embodiments, an adhesive is used to attach the output interface 218 to the device 200, or to attach the locating ridge 400 to the output interface 218.

In another embodiment, the acne treatment device 200 includes a safety system, such as the safety system 160 described above with respect to FIG. 1. In one embodiment, the safety system includes circuitry and/or sensor that prevent light source activation until a safety condition is realized. For example, in some embodiments, the safety system includes a switch that is activated prior to enabling activation of the light source.

For example, the safety system can include, but is not limited to, a mechanical pressure switch, a contact switch, and/or an electrical switch such as a galvanic response, resistance or impedance switch. A galvanic response device is activated when brought into contact with a user's skin. The galvanic response device can prevent the device 200 from emitting light unless the device 200 is in contact with the user's skin. In addition, the galvanic response device can prevent light from leaking or being emitted from the device 200 when activated, e.g., flashed.

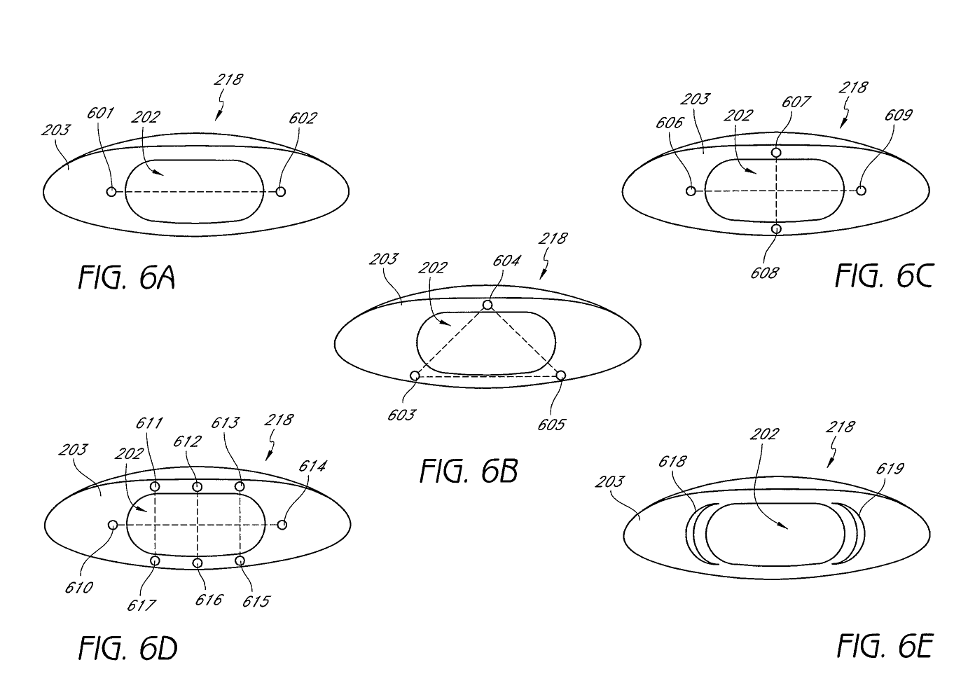

Embodiments of safety switch arrangements are illustrated by FIGS. 6A-E. In the various embodiments, safety switch contacts 601-619 (sometimes referred to as sensors, or contact sensors) are arranged around the emission channel 202. In some embodiments, the contact arrangement is referred to as a contact array. For example, in the embodiment illustrated in FIG. 6A, two contacts 601, 602 are positioned arranged on opposite side of the emission channel 202. Until the patient's skin comes in contact the switch contacts 601, 602, the switch is open and the safety system 160 will not allow the device 100 to flash. Once the skin comes in contact with the contacts 601, 602 the switch will close and the user may activate the device 200 light source.

FIGS. 6B-D illustrate contact configurations employing three, four and eight contacts, respectively. The dotted lines represent linear paths between various combinations of contacts, which when brought into contact with the user's skin, will close the safety switch and enable device 200 activation. For example, in FIG. 6C, the switch will close when either of two contact pairs 606, 609 or 607, 608 are brought into contact with the user's skin. In one embodiment, the linear paths at least partially traverse the emission channel 202 defined by the user interface 218. By assuring that such contacts are touching the user's skin prior to activation, the device 200 can determine whether the entire planar surface traversing the emission channel 202 is in contact with the user's skin, or if it is inclined at an angle with respect to the user's skin.

The dotted lines serve as possible combinations only and are not meant to limit the number of combinations possible in other embodiments. For example, in other embodiments, simultaneous contact with contacts 606 and 607 may also enable device 200 activation.

The embodiments of FIGS. 6A-D include contacts 601-614 having circular cross-sectional areas. However, in various embodiments, the safety switch contacts 601-614 may be shaped differently. For example, FIG. 6E illustrates a safety switch including crescent shaped contacts 618, 619 which are shaped to generally conform to the shape of window 202. contact shape can be selected to optimize user skin contact for a particular application.

As described above, in some embodiments, the safety switch includes various types of switches including analog- and digital-type electrical switches. The skin is an electrical conductor and therefore has a corresponding resistance. Therefore, the safety system can not only determine contacts are touching skin, but by analyzing the resistance measurements (or signals) obtained from various contacts, the safety system can determine the angle at which the user interface 218 is aligned or tilted with respect to a treatment area on the user's skin.

In other embodiments, the safety system uses the contact sensors or sensor arrays as digital switches. For example, in one embodiment, the safety system monitors the resistance between two contacts, and enables activation of the light source only when the resistance between the appropriate safety switch contacts falls beneath a certain threshold.

For example, with respect to FIG. 6A, when at least one of the contacts 601, 602 is not in contact with the skin, the resistance between the contacts 601, 602 is very large and the switch is open. In this situation, the device 200 will remain disabled by the safety system. However, when the contacts 601, 602 are both brought in contact with the skin, the resistance between them drops to an amount corresponding to resistance of the skin, and the switch will close. The safety system will cause activation, e.g. flashing, of the device 100 to be enabled. Moreover, in certain embodiments, the switch is configured to close when brought into contact with the skin but not when brought into contact with other conductive surfaces having different electrical characteristics.

One advantage of using electrical impedance sensors as contact sensors is that the safety system cannot be fooled into thinking that it is in contact with skin by merely pressing down on a mechanical switch. This functionality improves device 200 safety, as it prevents the device 200 from being activated when not contacting skin, which could lead to light emission into a user's or other party's eyes. Product safety is further enhanced by providing two or more sensors, as discussed above.

In other embodiments, the safety switch includes mechanical switches, such as a mechanical pressure switch that closes when a certain amount of pressure is detected by the switch. For example, with respect to FIG. 6A, if a certain threshold pressure is placed on the contacts 601, 602, the switch closes, and the safety system 160 causes activation, of the device 200.

The illustrated embodiments include multiple contacts 601-619, at least two of which are in contact with the skin to enable activation of the device 200. However, in other embodiments, there may be only one contact or only one contact may need to be in contact with the skin to enable activation of the device. For example, in certain embodiments, a single contact may be placed along rim 204 or locating ridge 400 or portions thereof. Moreover, while the illustrated embodiments show the contact or contacts 601-619 disposed on the surface 203, in other embodiments, the contact or contacts 601-619 may be located in various other portions of output interface 218. For example, the contact or contacts 601-619 may be located underneath the surface 203, on the rim 204, or on or within locating ridge 400.

In various embodiments, the acne treatment device 200 is configured to limit operability to a predetermined event. For example, the device 200 is generally configured such that is not usable after a certain amount of time, light exposure, light pulses, activations, etc. After the predetermined event occurs, the device 200 can be re-activated. For example, in some cases, the device 200 is re-activated by entering a validation code, or by replacing a part, such as the light source or power supply. In other embodiments, the device 200 is re-activated by downloading an activation code or activation signal from a remote location, such as over the Internet.

In one embodiment, the controller is configured to limit operability of the optical device 200. For example, the controller can be configured to prevent the optical device 200 from emitting light after a predetermined event. In one embodiment, the controller prevents optical device 200 operation after a predetermined number of light flashes, or light emissions are produced (e.g., 50, 100, 250, 500, or 1000 light flashes). In other embodiments, the controller prevents optical device 200 operation after a predetermined time of total light emission (e.g., 5, 10, 15, 30, 60, or 120 minutes). In other embodiments, the controller prevents optical device 200 operation after a predetermined event, such as a predetermined number of device-to-skin contacts (e.g., 50, 100, 250, 500, or 1000 contacts).

In general, the lifetime of the light source can be predetermined and/or set to expire after a preset number of flashes, by mechanical and/or electronic operations, including software and firmware. Such software and/or firmware can be included in the device 200, controller, and/or housing 210. In one embodiment, after reaching the preset maximum number of flashes, the device 200 is re-activated by removing the light source and replacing it with a new light source.

In other embodiments, the device 200 includes an audible signal that warns the user that the system is ready to flash. If within a preset period of time the unit is not placed on to the skin so that the pressure/contact switch is activated the unit discharges, and the flash charge is lost.

In other embodiments, the device 200 includes a timer, such as a timing circuit. The timer is configured to prevent rapid flashing by the user. For example, the timer can provide a delay of about 1, 5, 10, 30, or more than 30 seconds between flashes or light emissions from the light source. The timing circuit can be provided as discreet circuitry and/or implemented with the controller.

In other embodiments, the device 200 includes an indicator, such as an LED, display, icons, microphone, and/or other indicator. In one embodiment, the device 200 emits an audible signal that warns the user that the power is too low on the unit to use.

In other embodiments the device 200 includes at least one security device capable of determining whether the device 200 remains a compliant device. For example, the security device may be configured to assist in the determination of whether the power source, light source or output interface 218 are compliant with device 200. The security device may be, for example, a 20K EEPROM well known to those of skill in the art and capable of performing various diagnostic and control functions.

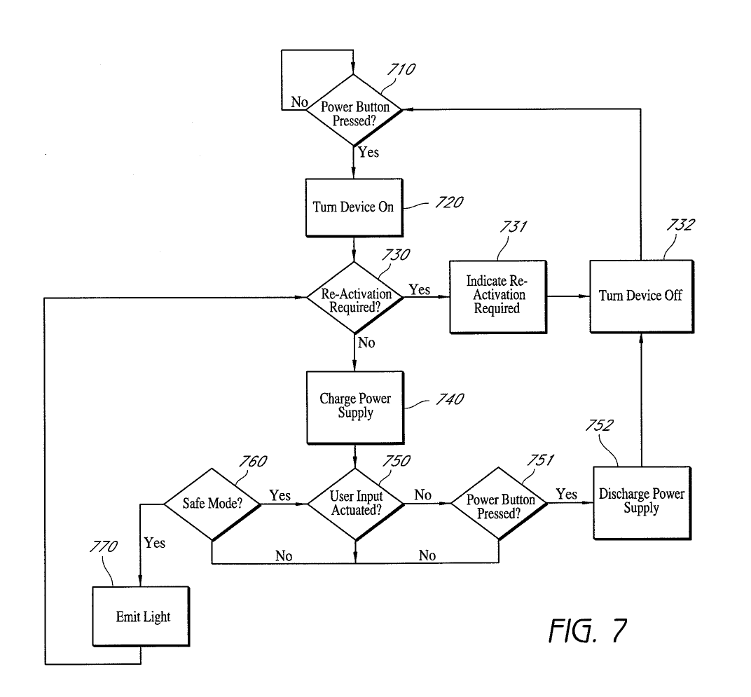

FIG. 7 shows one embodiment of using an acne treatment device. A power button is pressed to turn on the device at step 710. The device is then turned on at step 720. The method then determines if a re-activation of the device is required at step 730. For example, the device determines if a predetermined number of light flashes have already been provided by the device. If so, the device indicates that re-activation is required at step 731, and then stops by turning the device off at 732. Use of the device and light source is prevented.

However, if not, the method charges the power supply at step 740. For example, the method charges a capacitor. When charged, the method indicates that the device is ready to be used. The method waits until a user input interface is actuated at step 750. For example, the method waits until the user presses button 207. Prior to actuation, the method monitors the power button to determine if it is pressed again at step 751. If so, the device discharges the power supply and shuts off at step 750.

Otherwise, when the user input interface is actuated, the method checks to see if the safety system is in safe mode at step 760. For example, the method checks to see if the appropriate contacts of the safety switch are in contact with the user's skin. If not, the method waits until the unit is in safe mode and the user input interface is actuated again at step 750. If so, the method causes the device to emit a therapeutic light dosage to the user's skin through the output interface at step 770. After the light is emitted, the method returns to step 730 to determine whether re-activation of the device is required.

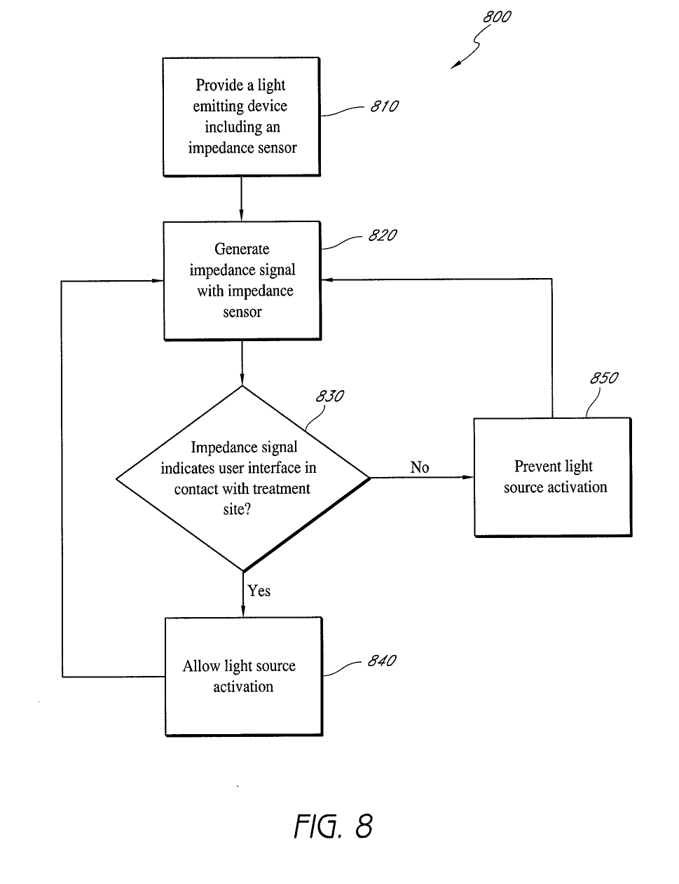

FIG. 8 shows a method 800 of treating acne with an acne treatment device according to an embodiment of the disclosure. The method 800 begins at step 810, in which a light emitting device is provided. The light emitting device is the device 100 described above with respect to FIG. 1. In other embodiments, the light emitting device is the device 200 described above with respect to FIG. 2, or some other light emitting device. The light emitting device includes a light source configured to generate optical energy having a wavelength in a range of from about 400 nm to about 1100 nm. In some embodiments, the light source is the light source 120, described above with respect to FIG. 1, or another light source.

The device provided by method 800 further includes a user interface configured to provide a transmission pathway for the optical energy from the light source to a treatment area generally along a beam propagation axis. The transmission pathway may be, for example, transmission path 202, described above with respect to FIG. 2. For example, in some embodiments, the user interface may be the user interface 150 described above with respect to FIG. 1, the user interface 218 described above with respect to FIG. 2, or some other user interface. The user interface includes an electrical impedance sensor, such as, for example, any of the electrical impedance sensors described above, or some other impedance sensor. In other embodiments, the impedance sensor may be another impedance sensor. The device also includes a controller, for example, processor 140 described above with respect to FIG. 1, which is in electrical communication with the light source and electrical impedance sensor.

The method 800 includes generating an impedance signal with the electrical impedance sensor at step 820. At decision step 830, the method 800 determines whether the user interface is in contact with the treatment site. If the user interface is in contact with the treatment site, the method 800 allows activation of the light source and generation of the optical energy at step 840. The method 800 then returns to step 820. On the other hand, if at step 830 the method 800 determines that the user interface is not in contact with the treatment site, the method 800 prevents activation of the light source at step 850. The method 800 then returns to step 820.

The method 800 may include additional steps not shown in FIG. 8. For instance, the method 800 may include activating the light source, generating the optical energy and directing the optical energy to a treatment area. The method 800 may further include filtering the optical energy prior to delivery to the treatment area. The filtering step may include removing energy having a wavelength outside of a range of from about 400 nm to about 700 nm or by removing energy having a wavelength below about 400 nm. In other embodiments, the method 800 also includes generating additional optical energy with a second light source. The additional optical energy may include mostly infrared energy. Method 800 may further include illuminating the treatment area with an illumination light source, such as, for example, any of the illumination light sources described above.

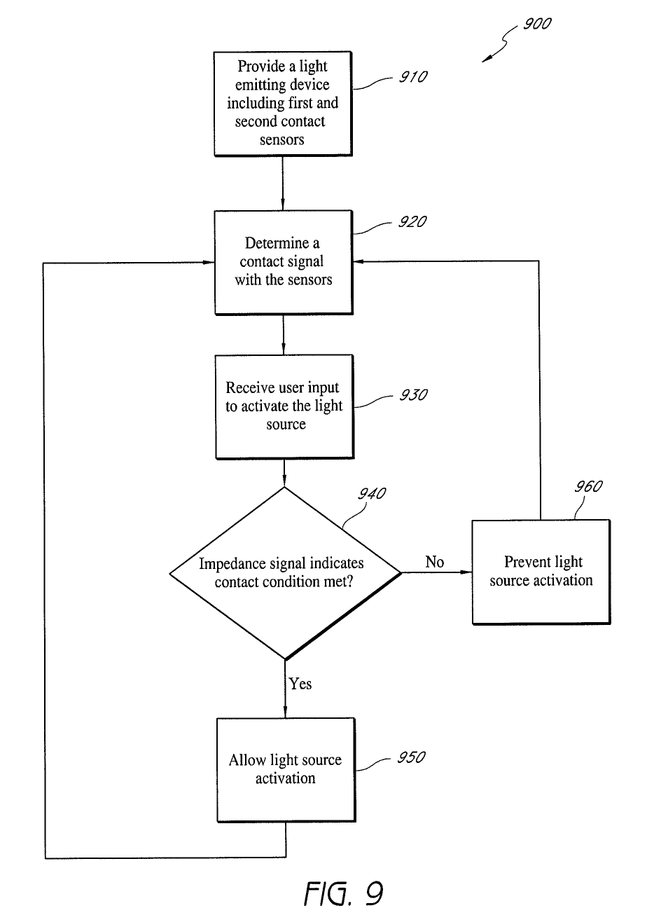

FIG. 9 shows a method 900 of treating acne with an acne treatment device according to an embodiment of the disclosure. The method 900 begins at step 910, in which a light emitting device is provided. The light emitting device may be the device 100 described above with respect to FIG. 1, the light emitting device 200 described above with respect to FIG. 2, or some other light emitting device. The light emitting device includes a light source configured to generate optical energy having a wavelength in a range of from about 400 nm to about 1100 nm. In some embodiments, the light source is the light source 120, described above with respect to FIG. 1, or another light source.

The acne treatment device further includes a user interface configured to provide a transmission pathway of the optical energy from the light source to a treatment area generally along a beam propagation axis. For example, in some embodiments, the user interface may be the user interface 150 described above with respect to FIG. 1, the user interface 218 described above with respect to FIG. 2, or some other user interface. The user interface is configured to provide a transmission pathway of the optical energy from a light source to a treatment area and includes at least a first and contact sensor spaced apart from each other. The transmission pathway may be, for example, transmission path 202, described above with respect to FIG. 2. A linear path from the first contact sensor to the second contact sensor at least partially traverses the transmission pathway. For example, in some embodiments, the contact sensors are the contact sensors described above with respect to FIGS. 6A-6E. For example, with respect to FIG. 6A, the first and second contact sensors may be contacts 601, 602. Moreover, in one embodiment, the linear path is the linear path represented by the dashed line of FIG. 6A.

The method 900 further includes determining a contact signal with the contact sensor information provided by the first and second contact sensors at step 920. The method 900 then receives user input at step 930 indicating that the user is attempting to activate the light source. For example, the user input may be the user pressing button 207 or some other button or user input mechanism. At decision step 940 the method 900 determines whether a contact condition is met. For example, the method 900 uses the contact signal to determine whether the first and second contact sensors defining the linear path are in contact with the treatment area. If the contact condition is met, the device allows light source activation at step 950. The method 900 then returns to step 920. On the other hand, at step 960, if the contact condition is not met, the method 900 prevents activation of the light source. The method 900 then returns to step 920.

The method 900 may include additional steps not shown in FIG. 9. For instance, the method 900 may include activating the light source when the contact signal indicates that contact condition is met. The method 900 may further include receiving a user input to determine if a button has been pressed or released. The method 900 may also include filtering optical energy after activating the light source. The filtering step may include removing energy having a wavelength outside of a range of from about 400 nm to about 700 nm, or removing energy having a wavelength below about 400 nm. In other embodiments, the method 900 also includes generating additional optical energy with a second light source. The additional optical energy may include mostly infrared energy. The method 900 may further include illuminating the treatment area with an illumination light source prior to activating the light source.

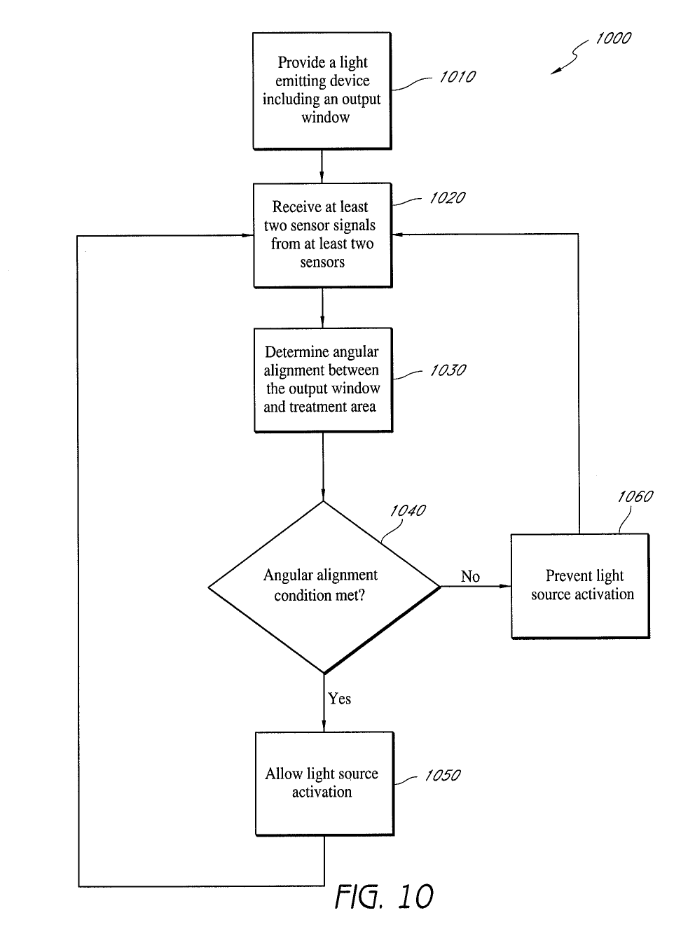

FIG. 10 shows a method 1000 of treating acne with an acne treatment device according to another embodiment of the disclosure. The method 1000 begins at step 1010, in which a light emitting device is provided. The light emitting device includes an output window. The output window may be any of the output windows described above, or another output window. The light emitting device may be the device 100 described above with respect to FIG. 1, the light emitting device 200 described above with respect to FIG. 2, or some other light emitting device. The light emitting device includes a light source configured to generate optical energy having a wavelength in a range of from about 400 nm to about 1100 nm. In some embodiments, the light source is the light source 120, described above with respect to FIG. 1, or another light source.

The device further includes a user interface configured to provide a transmission pathway of said optical energy from said light source to a treatment area generally along a beam propagation axis. For example, in some embodiments, the user interface may be the user interface 150 described above with respect to FIG. 1, the user interface 218 described above with respect to FIG. 2, or some other user interface. The user interface includes at least two sensors.

At step 1020 the method 1000 receives at least two sensor signals from the sensors. The sensors may be any of the sensors described above or other sensors. Based on the sensor signals, the method 1000 determines the angular alignment between the output window and a treatment area at step 1030. At decision step 1040, the method 1000 determines whether an angular alignment condition is met. For example, the method 1000 determines whether the device is substantially parallel to the surface of the treatment area. In another embodiment, the method 1000 may determine if the output window and treatment area are substantially in contact or if the angle between them is less than a predetermined value. If the angular alignment condition is met, the method 1000 allows activation of the light source at step 1050. The method then returns to step 1020. On the other hand, if the angular alignment condition is not met, the method 1000 prevents light source activation at step 1060. The method then returns to step 1020. The method 1000 may further include illuminating the treatment area with an illumination light source. The illumination light source may include any of the illumination light sources described above.

Although the foregoing invention has been described in terms of certain preferred embodiments, other embodiments will be apparent to those of ordinary skill in the art from the disclosure herein. Moreover, the described embodiments have been presented by way of example only, and are not intended to limit the scope of the inventions. Indeed, the novel methods and systems described herein may be embodied in a variety of other forms without departing from the spirit thereof. Accordingly, other combinations, omissions, substitutions and modifications will be apparent to the skilled artisan in view of the disclosure herein.

What is claimed is:

1. A light emitting device for providing therapy to a user, comprising:
a light source, configured to generate optical energy having a wavelength in a range of from about 400 nm to about 1100 nm;
a user interface, configured to be placed into contact with a treatment area on a user's body and configured to transmit said optical energy from said light source to said treatment area generally along a beam propagation axis, wherein said user interface comprises an electrical impedance sensor configured to determine when said user interface is contacting said treatment area; and
a controller, configured to receive at least one sensor signal from said electrical impedance sensor, wherein said controller is configured to prevent activation of said light source based upon said at least one sensor signal.

2. The light emitting device of claim 1, wherein said light source comprises a flashlamp.

3. The light emitting device of claim 1, wherein said light source comprises an LED.

4. The light emitting device of claim 1, wherein said wavelength is in a range of from about 400 nm to about 700 nm.

5. The light emitting device of claim 1, further comprising a transmission window configured to filter said optical energy prior to transmission to said treatment area.

6. The light emitting device of claim 1, further comprising a second light source configured to generate second optical energy having a second wavelength.

7. The light emitting device of claim 6, wherein said second wavelength comprises an infrared wavelength.

8. The light emitting device of claim 6, wherein said second wavelength comprises a blue wavelength.

9. The light emitting device of claim 6, wherein the second light source comprises an LED.

10. The light emitting device of claim 1, further comprising an aiming beam, configured to illuminate the treatment area prior to activation of said light source.

11. The light emitting device of claim 1, further comprising a user input configured to activate said light source.

12. The light emitting device of claim 11, wherein said user input comprises a button having a button depression axis, wherein said button depression axis is substantially aligned with said beam propagation axis.

13. The light emitting device of claim 1, wherein said user interface defines a transmission pathway through an opening in said user interface, said user interface further comprises a locating ridge positioned at least partially around said opening and configured to provide tactile feedback to a user regarding the position of the opening.

14. The light emitting device of claim 13, wherein said locating ridge extends around the entire opening.

15. A light emitting device for providing therapy to a user, comprising:
a light source, configured to generate optical energy having a wavelength in a range of from about 400 nm to about 1100 nm; and
a user interface, configured to be placed into contact with a treatment area on a user's body and configured to transmit said optical energy from said light source to said treatment area, said user interface defining a transmission pathway of said optical energy from said light source to said treatment area,
wherein said user interface comprises a first contact sensor and a second contact sensor spaced apart from said first contact sensor, and
wherein a linear path from said first contact sensor to said second contact sensor at least partially traverses said transmission pathway.

16. The light emitting device of claim 15, further comprising a controller, wherein said controller is configured to activate said light source only when both said first and second contact sensors are in contact with said treatment area.

17. The light emitting device of claim 15, wherein said first and second contact sensors comprise first and second impedance sensors.

18. The light emitting device of claim 15, wherein said light source comprises a flashlamp.

19. The light emitting device of claim 15, further comprising a filter configured to filter said optical energy prior to delivery to said treatment area.

20. The light emitting device of claim 15, further comprising a second light source configured.

21. The light emitting device of claim 20, wherein said second light source comprises an LED.

22. A light emitting device for providing therapy to a user, comprising:
a light source, configured to generate optical energy having a wavelength in a range of from about 400 nm to about 1100 nm; and
a user interface, configured to be placed into contact with a treatment area on a user's body and configured to transmit said optical energy from said light source to said treatment area, said user interface comprising an output window, and at least two contact sensors; and
a controller, configured to determine an angular alignment between said output window and said treatment area prior to delivering optical energy to said treatment area.

23. The light emitting device of claim 22, wherein said controller permits activation of said light source when said output window is determined to be substantially parallel to said treatment area.

24. The light emitting device of claim 22, wherein said controller permits activation of said light source when said output window is determined to be inclined with respect to said treatment area no more than about 22 degrees.

25. The light emitting device of claim 22, wherein said at least two contact sensors comprise at least two impedance sensors.

26. The light emitting device of claim 22, further comprising a second light source configured to generate blue light.

27. The light emitting device of claim 26, wherein said second light source comprises an LED.

28. A method of treating a physiological condition with optical energy, comprising:
providing a light emitting device, said light emitting device comprising:
a light source configured to generate optical energy having a wavelength in a range of from about 400 nm to about 1100 nm;
a user interface configured to provide a transmission pathway of said optical energy from said light source to a treatment area generally along a beam propagation axis, wherein said user interface comprises an electrical impedance sensor; and
a controller, in electrical communication with said light source and electrical impedance sensor;
generating an impedance signal with said electrical impedance sensor; and
preventing generation of said optical energy when said impedance signal indicates that said user interface is not in contact with said treatment site.

29. The method of claim 28, further comprising generating said optical energy and directing said optical energy to said treatment area.

30. The method of claim 29, further comprising filtering said optical energy prior to delivery to said treatment area.

31. The method of claim 30, wherein said filtering removes energy having a wavelength outside of a range of from about 400 nm to about 700 nm.

32. The method of claim 29, further comprising generating second optical energy with a second light source.

33. The method of claim 32, wherein said second optical energy comprises mostly infrared energy.

34. The method of claim 29, further comprising illuminating said treatment area with an illumination light source.

35. A method of treating a physiological condition with optical energy, comprising:
providing a light emitting device, comprising:
a light source, configured to generate optical energy having a wavelength in a range of from about 400 nm to about 1100 nm; and
a user interface configured to provide a transmission pathway of said optical energy from said light source to a treatment area, wherein said user interface comprises a first contact sensor and a second contact sensor spaced apart from said first contact sensor, wherein a linear path from said first contact sensor to said second contact sensor at least partially traverses said transmission pathway;
determining a contact signal with said contact sensors;
receiving a user input to activate said light source; and
activating said light source in response to said contact signal and user input.

36. The method of claim 35, wherein said activating comprises activating said light source when said contact signal indicates that said user interface is in contact with said treatment area and when said user input is activated.

37. The method of claim 35, wherein said receiving a user input comprises determining if a button has been pressed or released.

38. The method of claim 35, further comprising filtering said optical energy after activating said light source.

39. The method of claim 38, wherein said filtering removes energy having a wavelength outside of a range of from about 400 nm to about 700 nm.

40. The method of claim 35, further comprising generating second optical energy with a second light source.

41. The method of claim 40, wherein said second optical energy comprises mostly infrared energy.

42. The method of claim 35, further comprising illuminating said treatment area with an illumination light source prior to said activating.

43. A method of treating a physiological condition with optical energy, comprising:
providing a light emitting device, comprising:
a light source, configured to generate optical energy having a wavelength in a range of from about 400 nm to about 1100 nm; and
a user interface, configured to transmit said optical energy from said light source to a treatment area, said user interface comprising an output window, and at least two sensors; and
receiving at least two sensor signals from said at least two sensors;
determining an angular alignment between said output window and said treatment area based upon said at least two sensor signals.

44. The method of claim 43, further comprising enabling activation of said light source in response to said angular alignment.

45. The method of claim 44, wherein said enabling occurs only when said angular alignment indicates that said output window and said treatment area are substantially parallel.

46. The method of claim 44, wherein said enabling occurs only when said angular alignment indicates that said output window and said treatment area are substantially in contact.

47. The method of claim 43, further comprising illuminating said treatment area with an illumination light source, wherein said light emitting device further comprises said illumination light source.
